(19) United States
(12) Patent Application Publication
(10) Pub. No.: US 2014/0288567 A1
(43) Pub. Date: Sep. 25, 2014

(54) PEDICLE SCREW DRIVER

(71) Applicant: ROYAL OAK INDUSTRIES, Bloomfield Hill, MI (US)

(72) Inventor: Matthew Kroll, Oakland Twp, MI (US)

(73) Assignee: ROYAL OAK INDUSTRIES, Bloomfield Hill, MI (US)

(21) Appl. No.: 14/202,916

(22) Filed: Mar. 10, 2014

Related U.S. Application Data
(60) Provisional application No. 61/792,836, filed on Mar. 15, 2013.

(51) Int. Cl. A61B 17/88

(52) U.S. Cl. CPC A61B 17/8886
USPC 606/104

(57) ABSTRACT

A pedicle screw driver is provided and includes an inner shaft and outer shell. The shell includes a sidewall defining an inner cavity and has a slot formed on the sidewall. The slot includes a narrowed slot portion. The inner shaft includes a narrowed shaft portion. The narrowed shaft portion is sized to fit through the narrowed slot portion, but not other wider portion of the slot. The shell includes apertures that can permit gas and fluids to access the inner cavity of the outer shell.

### FIELD

The embodiments disclosed herein relate to tools for assemblies used to stabilize spinal segments or other bone structures.

### BACKGROUND

Spinal stabilization assemblies are used in order to secure adjacent sections of the vertebral column. Stabilization procedures are performed in spinal fusion surgeries, where an intervertebral disc is wholly or partially removed and the adjacent vertebra are fused with bone to combine adjacent segments of the vertebral column, creating a larger singular bone structure. This type of treatment can be used to treat spinal deformity, injury or disease.

Pedicle screws have been widely used in such treatments for over forty years. Pedicle screws serve as anchor points in adjacent or nearby vertebrae that can then be connected with a rod or the like to stabilize the spinal segment while the adjacent vertebra fuse. Such spinal assemblies largely use polyaxial screw systems to accomplish vertebral connection. Polyaxial screws generally comprise a spherical head enclosed in a housing that is able to pivot at any angle relative to the polyaxial screw. Polyaxial screw systems are described in U.S. Pat. No. 5,207,678 to Harms et al and U.S. Pat. No. 5,690,630 to Errico et al.

Because the polyaxial pedicle screw serves as the anchor upon which the connecting components rely for their orientation, it is important that the pedicle screw is secured in a proper position with respect to the other components of the assembly. This allows physicians to create a secure and dependable fused spinal segment.

Various instruments and tools have been developed to insert and assemble pedicle screw assemblies in a reliable and safe manner.

### SUMMARY

According to some embodiments, a screwdriver is disclosed. The screwdriver comprises an outer shell comprising a sidewall defining an inner cavity and having a slot formed therein, the slot comprising a first slot portion and a second narrowed slot portion; and an inner shaft, the shaft comprising a first shaft portion and a second narrowed shaft portion, wherein the second narrowed shaft portion is sized to fit through the second narrowed slot portion but not the first slot portion.

According to some embodiments, a method of assembling a pedicle screw implant driver is disclosed. The method comprises aligning a narrowed slot portion of an outer shell with a narrowed shaft portion of an inner shaft; first inserting the narrowed shaft portion into the outer shell portion; and then inserting any additional portion of the inner shaft into the outer shell.

According to some embodiments, a pedicle screw implant driver is disclosed. The driver comprises an outer shell; an inner shaft; and means for manually inserting and removing the inner shaft into the outer shell and retaining the inner shaft therein without the use of any tool.

### BRIEF DESCRIPTION OF THE FIGURES

FIGS. 1A-1C schematically illustrate example embodiments of components of a pedicle screw implant driver.

FIG. 2 schematically illustrates an example embodiment of a component of a pedicle screw driver.

FIG. 3 schematically illustrates an example embodiment of a component of a pedicle screw driver.

FIG. 4 schematically illustrates an example embodiment of a pedicle screw implant driver.

FIGS. 5A and 5B illustrate side views of the components of FIGS. 2 and 3.

FIG. 6 schematically illustrates a cross-sectional view of a pedicle screw assembly.

### DETAILED DESCRIPTION

The embodiments disclosed herein relate to tools for assemblies for spinal stabilization.

Embodiments will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments disclosed herein. Furthermore, embodiments disclosed herein may include several novel features, no single one of which is solely responsible for its desirable attributes or which is essential to the embodiments herein described.

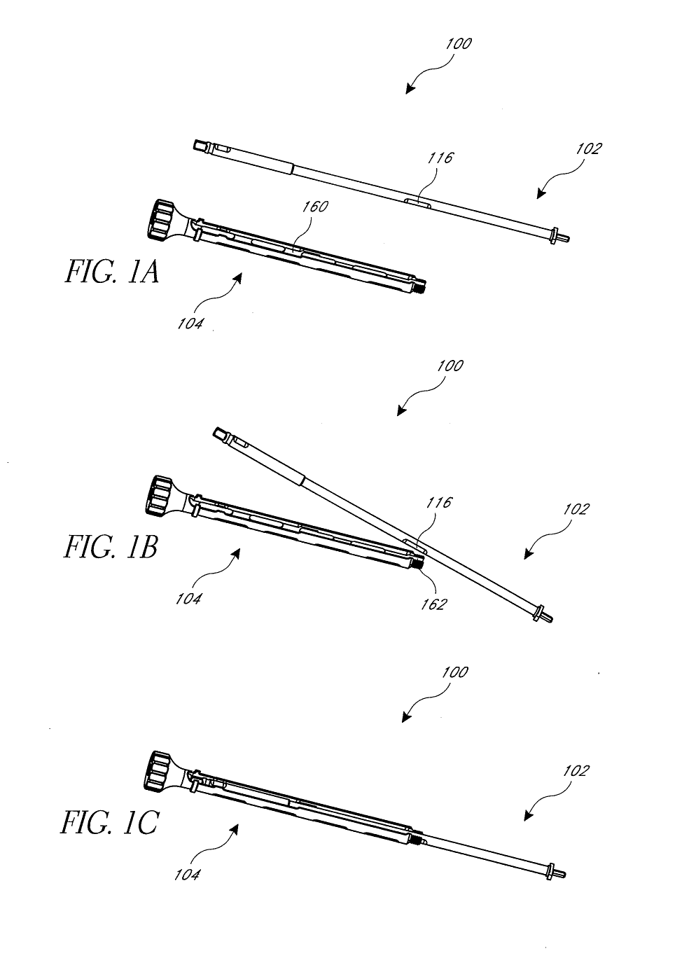

Referring now to FIGS. 1A-1C, a pedicle screw implant driver 100 according to one embodiment is shown. The driver 100 comprises an inner shaft 102 and an outer shell 104. The outer shell comprises a sidewall defining a slot 160. FIG. 1B depicts a first narrowed shaft portion 116 of the inner shaft being aligned with a first narrowed slot portion 162 of the slot 160. The first narrowed portion 116 of the shaft 102 may comprise a width that is smaller than the width of the rest of the shaft 102. The narrowed portion 116 may be sized to fit within the first narrowed portion 162 of the slot, while the remainder of the shaft may not be sized to fit within the first narrowed portion of the slot, but may fit within the rest of the slot. The first narrowed shaft portion 116 may be inserted into the first narrowed slot portion 162. The remainder of the shaft 102 may then be inserted into the shell 104 because the remainder of the shaft fits through the remainder of the slot. In some embodiments, the inner shaft 102 and the slot 160 comprise additional narrowed portions. After insertion of the narrow shaft portions into the narrowed slot portions, the remainder of the shaft 102 may be inserted into the shell 104. FIG. 1C depicts the inner shaft 102 completely inserted into the outer shell 104. The inner shaft 102 and outer shell 104 are described in more detail below.

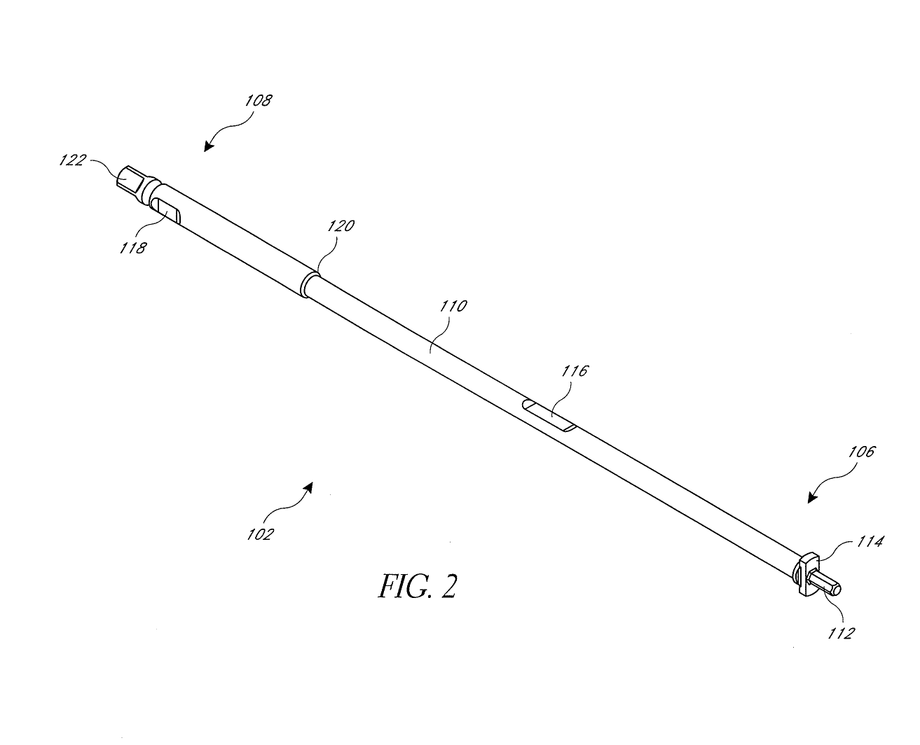

Moving to FIG. 2, an embodiment of an inner shaft 102 is depicted. The inner shaft 102 comprises a proximal end 106 and a distal end 108. A main shaft portion 110 may extend between the proximal end 106 and the distal end 108. The proximal end 106 of the shaft 102 may include a drive coupler 112. The drive coupler may be configured to be coupled to a head of a pedicle screw. For example, the drive coupler 112 may comprise a shape that is sized to fit in a recess at the head of a pedicle screw. The drive coupler is shown as comprising a hexagonal shape, but other shapes (e.g., square, rectangular) are also possible. The drive coupler 112 is shown as a male coupler configured to be coupled to a female screw; however the coupler may also be a female coupler configured to be coupled to a male screw. Other mechanisms for coupling (e.g., threads) are also possible.

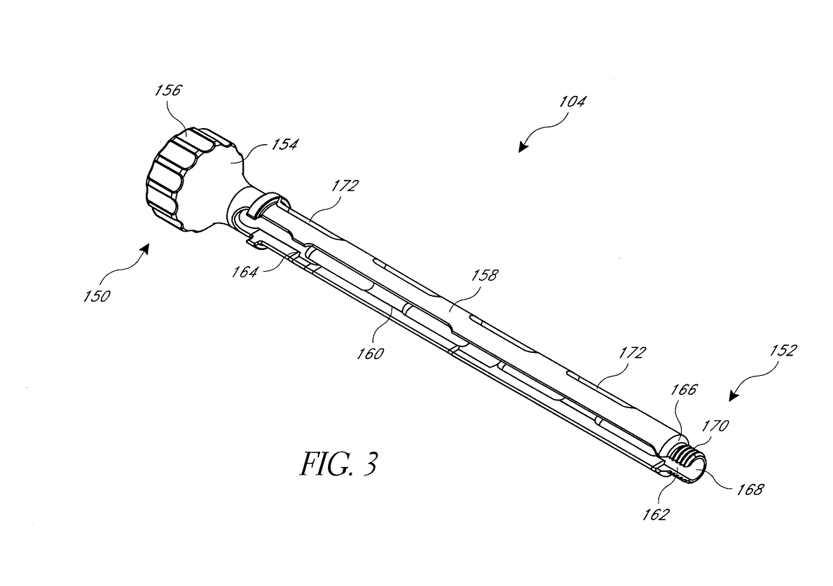

The proximal end 106 of the shaft 102 may also comprise a shaft shoulder 114. The shaft shoulder 114 may rest upon or near the top of a head of a pedicle screw when the coupler 112 is engaging the pedicle screw. The shaft shoulder 114 comprises a width greater than the width of the drive coupler and the remainder of the shaft 102. When the shaft 102 is inserted within the cavity 168 (as shown in FIG. 3) of the shell 104, the shaft shoulder 114 may prevent the shaft 102 from extending proximally through the shell 104.

The shaft 102 may comprise a narrowed portion 116 along the middle shaft portion 110. The narrowed portion 116 has a portion that is narrower that the rest of the shaft 110. The narrowed potion 116 in FIG. 2 is shown as being narrower in only certain portions along the circumference of the shaft. In some embodiments, the narrowed portion 116 is narrowed around the entire circumference of the shaft 110. The narrowed portion 116 may be positioned at a different location along the shaft 102 than what is shown in FIG. 2.

The shaft 102 may include a second narrowed portion 118. The narrowed potion 118 in FIG. 2 is shown as being narrower in only certain portions along the circumference of the shaft. In some embodiments, the narrowed portion 118 is narrowed around the entire circumference of the shaft 110. The first narrowed portion 116 is shown as being longer than the second narrowed portion 118. In some embodiments, the second narrowed portion 118 is longer than the first narrowed portion 116. In some embodiments, the second narrowed portion 118 and the first narrowed portion 116 are the same size. The second narrowed portion 118 may be positioned at a different location along the shaft 102 than what is shown in FIG. 2.

The shaft 102 comprises an expanded portion 120. The expanded portion 120 may be too large to fit through a shoulder portion of the outer shell 104. The expanded portion 120 may help to keep the shaft 102 from fully extending through the shell 104.

The first narrowed portion 116 and the second narrowed portion 118 can be at different angles from one another as shown in FIG. 2. The first narrowed portion 116 and the second narrowed portion can be orthogonal in some embodiments. In some embodiments, the first narrowed portion 116 and the second narrowed portion 118 can be offset by between about 0° and about 90°.

In some embodiments, the proximal end 108 of the shaft 102 comprises a drive coupler 122. The drive coupler 122 may be configured to be coupled to a tool that a physician can use to more easily manipulate the shaft 102. The drive coupler 122 is shown as a male coupler shaped to mate with a female tool. The female tool may comprise a recess shaped to mate with the drive coupler 122. In some embodiments, the drive coupler 122 can be a female coupler. Other shapes and methods of coupling (e.g., threads) for the drive coupler 122 are also possible.

In some embodiments, the shaft 102 is between about 8 inches and about 13 inches long. In some embodiments, the shaft 102 is between about 9 inches and about 12 inches long. In some embodiments, the shaft 102 is about 11 inches long. In some embodiments, the shaft 102 may be shorter or longer than the stated dimensions.

Moving to FIG. 3, an embodiment of an outer shell 104 is shown. The outer shell 104 comprises a proximal end 150 and a distal end 152. The shell 104 comprises a central bore 168 extending from the proximal end 150 to the distal end 152. The central cavity 168 is shown as circular, but other shapes (e.g., square) are also possible.

The outer shell 104 comprises a knob 154 positioned at or near the proximal end 150. In some embodiments, the knob 154 is a greater diameter than the remainder of the outer shell 104. This greater diameter may allow the knob 154 to be easily manipulated by a physician using the driver 100. The knob 154 may comprise ridges 156 or other texturing which may allow for a physician to grip the tool better.

The outer shell 104 may comprise a sidewall 158 extending between the proximal end 150 and the distal end 152. In some embodiments, the sidewall 158 defines a slot 160 extending at least partially along the length of the sidewall. In some embodiments, the slot 160 extends substantially along the length of the sidewall 158. In some embodiments, as shown in FIG. 3, the slot 160 extends from a point proximal to the knob 154, near the proximal end 150 of the shell to the distal end 152 of the shell 104.

In some embodiments, the slot 160 comprises a first narrowed slot portion 162. The first narrowed slot portion may comprise a width narrower than a width of another portion of the slot 160. The first narrowed slot portion 162 may be sized to permit passage of a first narrowed portion 116 of the inner shaft 102 therethrough. The first narrowed portion 162 may be sized so that certain portions of the shaft 102 (e.g., portions other than the narrowed portions) cannot be inserted therethrough.

In some embodiments, the slot 160 comprises a second narrowed slot portion 164. The second narrowed slot portion 164 may comprise a width narrower than a width of another portion of the slot 160. The second narrowed slot portion 164 may be sized to permit passage of a second narrowed shaft portion 118 therethrough. The second narrowed slot portion 162 may be sized so that certain portions of the shaft 102 (e.g., portions other than the narrowed portions) cannot be inserted therethrough.

The second narrowed slot portion 164 is shown as shorter than the first narrowed slot portion 162. In some embodiments, the second narrowed slot portion 164 is longer than the first narrowed slot portion 162. In some embodiments, the second narrowed slot portion 164 is about the same length as the first narrowed slot portion 162. In some embodiments, the first and second narrowed slot portions 162, 164 comprise different widths. In some embodiments, the first and second narrowed slot portions 162, 164 comprise the same widths. In some embodiments, the shell 104 comprises more than two narrowed slot portions of the same or different widths and/or lengths as the first and second narrowed slot portions.

The shell 104 may comprise a shoulder 166 near the distal end 152. The shoulder 166 may comprise a portion of the shell 104 defining a portion of the bore 168 that is narrower than other portions of the bore 168. The shoulder 166 may define a portion of the bore 168 sized to prevent a portion of the shell (e.g., the expanded portion 120) from passing therethrough. The shoulder 166 may help prevent the inner shaft 102 from fully extending through the distal end 152 of the shell 104.

The shell 104 may comprise threads 170 along an outer surface of the distal end 152. The threads 170 may allow the shell 104 to be coupled to a component of a pedicle screw assembly, for example, a cup that comprises threads along an inner surface. In some embodiments, the threads may be positioned on the inner surface of the distal end. In such embodiments, the threads may be coupled to a cup (or other screw assembly component) that comprises threads along an outer surface. While FIG. 3 shows threads as a coupling mechanism, other mechanisms are also possible.

In some embodiments, the shell 104 comprises apertures 172 in the sidewall 158. FIG. 3 depicts the shell 104 comprising eight apertures. In some embodiments, the shell 104 comprises between about one and about 15 apertures. In some embodiments, the shell 104 comprises greater than 15 apertures. The shell 104 may contain no apertures. The apertures 172 are shown as ovular, but other shapes are also possible. For example, the apertures 172 may be circular or ovular. In some embodiments, larger or longer apertures may allow better access to an interior of the shell 104.

In some embodiments, the apertures 172 may be sized and configured to allow access to gas or fluids (e.g., water or sterilization gas). Gas or fluids may enter the bore 168 of the shell 104 to clean or sterilize the interior of the shell 104. Many pedicle screw drivers currently available are two-part drivers with one component comprising an inner cavity that can be difficult to sufficiently clean. The apertures 172 allow the shell 102 to be cleaned and sterilized in spite of its comprising an inner bore or cavity. Furthermore, regulatory agencies may prefer a pedicle screw driver component containing apertures 172 as it may facilitate more thorough cleaning and sterilizing of the pedicle screw driver as compared to other pedicle screw drivers currently available.

In some embodiments, the shell 104 is between about 5 inches and about 11 inches long. In some embodiments, the shell 104 is between about 7 inches and about 9 inches long. In some embodiments, the shell 104 is about 8 inches long. Other dimensions for the shell are also possible.

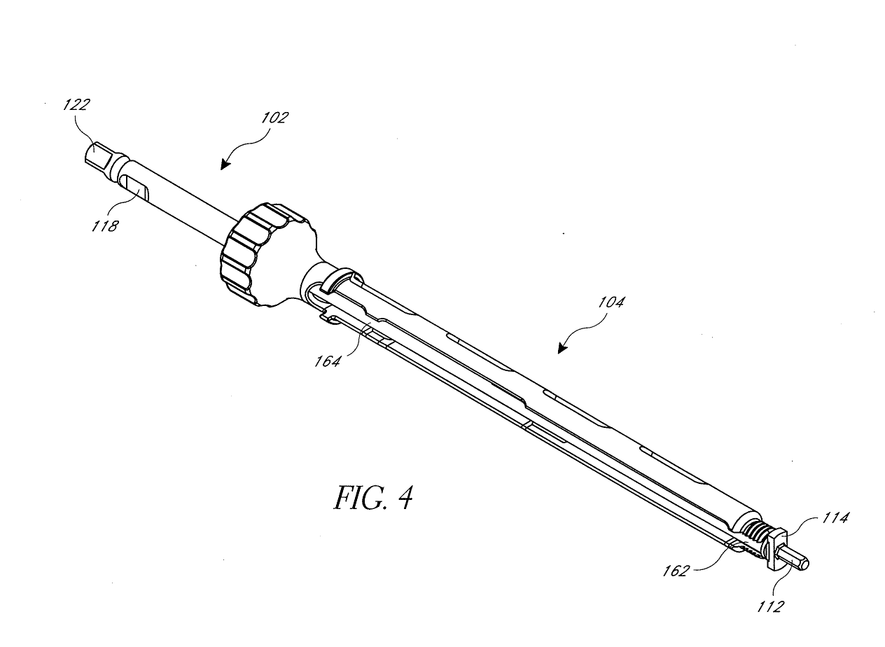

Moving to FIG. 4, an embodiment of an inner shaft 102 inserted within an outer shell 104 is depicted. The inner shaft 102 may be inserted into the inner cavity or bore of the outer shell by inserting the inner shaft 102 through the slot 160 by inserting the first narrowed portion 116 and the second narrowed portion 118 of the shaft 102 into the first narrowed portion 162 and the second narrowed portion 164 of the slot 160 of the shell 104. The first narrowed shaft portion 116 may first be inserted into the first narrowed slot portion 162. After insertion of the first narrowed shaft portion 116, a portion of the shaft 102 adjacent to the first narrowed shaft portion 116 can be inserted into the slot 160 through the wider portions of the slot 160. While inserting the shaft 102, the second narrowed slot portion 164 may be reached. In embodiments in which the second narrowed shaft portion 118 is at a different angle than the first narrowed shaft portion 116, such as that shown in FIG. 2, the shaft 102 can be rotated until the second narrowed shaft portion 118 is aligned with the second narrowed slot portion. Once the second narrowed shaft portion 118 is aligned with the second narrowed slot portion 164, the second narrowed shaft portion can be inserted through the second narrowed slot portion and the remainder of the shaft 102 may be inserted through the slot 160 into the inner cavity or bore 168. In embodiments in which the slot and shaft only include one narrowed shaft portion, the rotation may not be necessary. In embodiments of a driver comprising more than two narrowed portions, further rotation may be necessary to insert the additional narrowed shaft portions into the additional narrowed slot portions.

Once the inner shaft 102 is positioned within the cavity 168 of the shell 104, the shaft 102 is axially translatable within the shell 104. As discussed above, the inner shaft 102 may comprise an expanded portion 120 that prevents the inner shaft from fully extending through the distal end 152 of the shell 104. The shaft shoulder 122 may prevent the inner shaft from extending proximally into the shell 104.

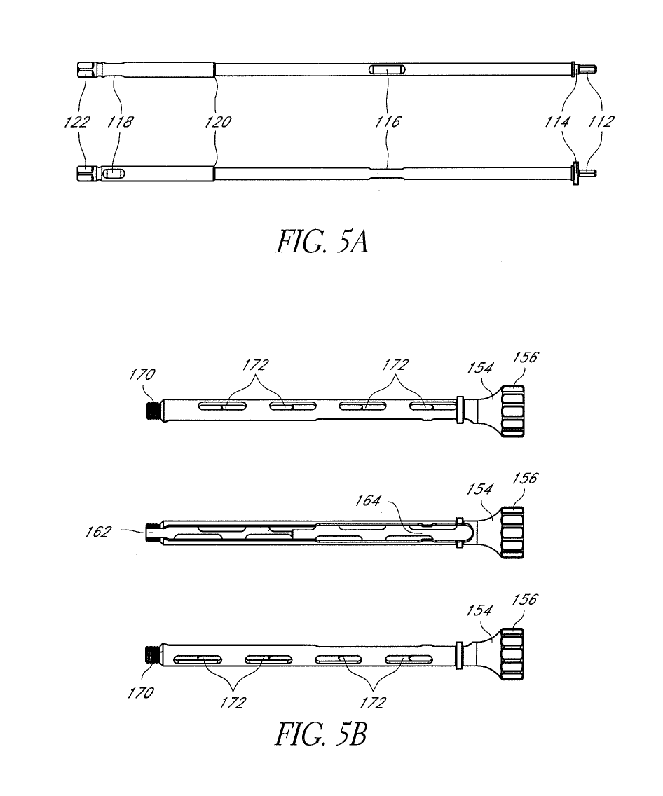

FIGS. 5A and 5B illustrate side views of the shaft and shell of FIGS. 2 and 3.

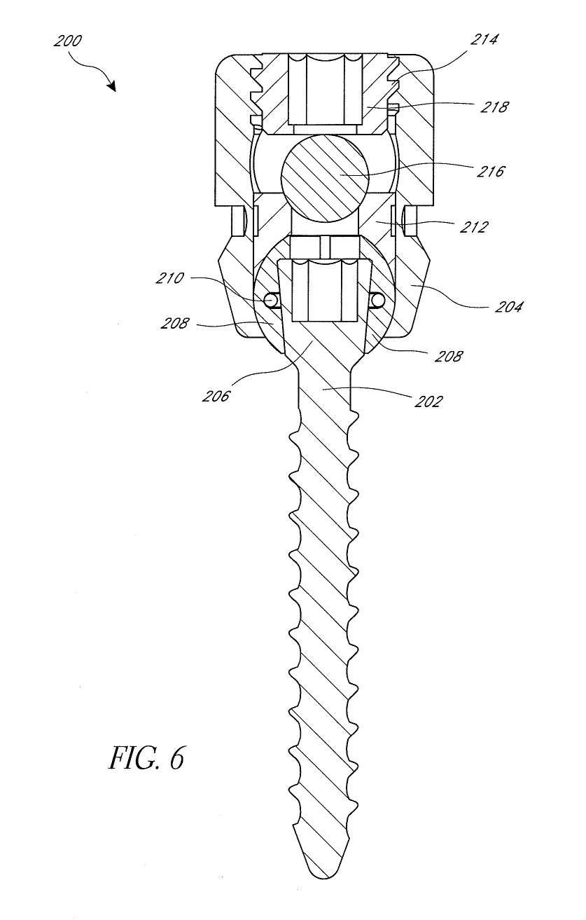

The pedicle screw driver 100 may be used to implant a pedicle screw assembly such as that shown in FIG. 6. FIG. 6 depicts a cross-sectional view of a pedicle screw assembly 200 comprising a fastener 202 extending through a bottom of a cup 204. The head 206 of the fastener 202 is surrounded by two keeper portions 208. A ring 210 is positioned between the head 206 of the fastener and the keeper portions 210. A saddle 212 is positioned above the keeper portions. The saddle comprises a bore (not shown) allowing access to the drive coupler (not shown) of the fastener 202. A rod 216 and set screw 218 are positioned above the saddle 212. Such a pedicle screw assembly is described in U.S. patent application Ser. No. 13/152,674, filed Jun. 3, 2011, the entirety of which is hereby incorporated by reference.

A physician may receive the pedicle screw assembly with the fastener 202 along with the ring 210 and keeper portions 208 positioned within the cup 204. The saddle 212 may also be positioned above the fastener 202. The physician may couple the drive coupler 122 of the inner shaft 102 to the drive coupler of the fastener 202. The physician may couple the shell to the cup 204 by mating the threads 170 at the proximal end 152 of the shell 104 to the threads 214 positioned at an inner surface of the top aperture (not shown) of the cup 204. The knob 154 can be used for ease in rotating the shell 104 to join the threads. Once the driver 100 is attached to the screw assembly by coupling the shell 104 and the cup 204, the physician may screw the fastener 204 into the bone by rotating the shaft 102. The physician may attach a tool to the drive coupler 122 at the proximal end 108 of the shaft 102 which may aid in rotating the shaft. Once the fastener 204 is sufficiently inserted into the bone, the shell 104 may be decoupled from the cup 204, and the driver 100 may be removed from the vicinity.

In some embodiments, the components described herein comprise a metal alloy. For example, they may comprise stainless steel alloys. Examples of stainless steel alloys include 17-4 SST and 465 SS. In some embodiments, the components of the driver 100 comprise a same material. In some embodiments, the components of the driver 100 comprise different materials.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A screwdriver comprising:
an outer shell comprising a sidewall defining an inner cavity and having a slot formed therein, the slot comprising a first slot portion and a second narrowed slot portion; and
an inner shaft, the shaft comprising a first shaft portion and a second narrowed shaft portion,
wherein the second narrowed shaft portion is sized to fit through the second narrowed slot portion but not the first slot portion.

2. The driver of claim 1, wherein the inner shaft comprises an upper portion with a larger diameter than a lower portion and wherein the outer shell comprises a lower portion with a smaller inner diameter than an upper portion.

3. The driver of claim 1, wherein a first end of the inner shaft is configured to be coupled to a pedicle screw.

4. The screwdriver of claim 1, wherein the outer shell comprises one or more openings in the sidewall sized and configured for sterilization gas access to the inner cavity.

5. The screwdriver of claim 1, wherein a first end of the outer shell comprises threads configured to be coupled to a cup of a screw assembly.

6. The screwdriver of claim 1, wherein a first end of the inner shaft extends through a first end of the outer shell when the inner shaft is inserted in the inner shell pedicle screw implant driver.

7. The driver of claim 1, wherein the shell comprises a second narrowed slot portion and the shaft comprises a second narrowed shaft portion.

8. The driver of claim 7, wherein the second narrowed shaft portion is positioned at a different angle than the first narrowed shaft portion.

9. The driver of claim 7, wherein the second narrowed shaft portion is orthogonal to the first narrowedshaft portion.

10. A method of assembling a pedicle screw implant driver, the method comprising:
aligning a narrowed slot portion of an outer shell with a narrowed shaft portion of an inner shaft;
first inserting the narrowed shaft portion into the outer shell portion; and
then inserting any additional portion of the inner shaft into the outer shell.

11. The method of claim 10, wherein inserting the additional portion comprises aligning a second narrowed slot portion of the outer shell with a second narrowed shaft portion of the inner shaft.

12. The method of claim 11, further comprising rotating the inner shaft after inserting the narrowed shaft portion into the narrowed shell portion to align the second narrowed slot portion with the second narrowed shaft portion.

13. A pedicle screw implant driver, the driver comprising
an outer shell;
an inner shaft; and
means for manually inserting and removing the inner shaft into the outer shell and retaining the inner shaft therein without the use of any tool.
